(19) United States
(12) Patent Application Publication
(10) Pub. No.: US 2022/0018849 A1
(43) Pub. Date: Jan. 20, 2022

(54) SERINE HYDROLASE PROFILING ASSAY IN BIOTHERAPEUTICS

(71) Applicant: Merck Sharp & Dohme Corp., Rahway, NJ (US)

(72) Inventors: Gregory C. Adam, Schwenksville, PA (US); Xuanwen Li, Belle Meade, NJ (US); Simon Letarte, Summit, NJ (US); Douglas D. Richardson, II, Collegeville, PA (US)

(21) Appl. No.: 17/355,547

(22) Filed: Jun. 23, 2021

Related U.S. Application Data
(60) Provisional application No. 63/043,966, filed on Jun. 25, 2020.

(51) Int. Cl. G01N 33/68; C12N 9/16; G01N 33/58; G01N 1/40

(52) U.S. Cl. CPC G01N 33/6848; G01N 1/4044; G01N 33/582; C12N 9/16

(57) ABSTRACT

The present disclosure describes a method of identifying serine hydrolase in a biological test sample obtained from protein production with a fluorophosphonate-containing probe. The present disclosure also provides a method of identifying one or more serine hydrolases in the biological test sample as causing PS-80 or PS-20 degradation.

### BACKGROUND OF THE INVENTION

Non-ionic surfactant polysorbate is an excipient in formulation solutions used as a shear protectant to stabilize biotherapeutics, prevent agitation-induced aggregation, and minimize surface adsorption.¹⁻³ Polysorbate-80 (PS-80) and polysorbate-20 (PS-20) are the most widely used polysorbates in the biopharma industry.²⁻³ The degradation of polysorbate could result in turbidity and potential formation of sub-visible particles, which may have a direct impact on product quality.¹⁻² Hence, polysorbate degradation can bring critical quality attribute (CQA) out of specification in several different ways. Polysorbate degradation is an industry-wide challenge for process and formulation development in biotherapeutics.¹⁻¹⁰ The challenge is increasing with higher titer fermentation batches and higher drug concentration in formulation development.¹⁰⁻¹¹ There are multiple mechanisms of polysorbate degradation, which can be grouped into two categories: oxidation and hydrolysis.¹³ The enzymatic hydrolysis pathway is primarily mediated by cleavage of an ester bond through lipases, esterases or other host cell proteins (HCPs). Those enzymes can co-purify with the therapeutic protein via specific or non-specific interactions.¹² Several lipases or esterases, such as PLBL2, LPL and LPLA2, have been reported to degrade PS-80 or PS-20 in biotherapeutics.^{4-7, 10}

Multiple analytical tools have been developed as part of an overall HCP control strategy.¹² Total HCP testing via traditional ELISA is currently considered the “gold standard”. An ELISA assay usually provides one summed value for total HCP content. Specific antibodies have been developed to known HCPs present in a sample.¹³⁻¹⁴ LC-MS based proteomics approach is evolving rapidly as an orthogonal assay for HCP characterization.¹⁵⁻¹⁹Proteomics can identify and quantify practically any individual HCP in the entire secretome to guide process development. Absolute quantification of individual HCPs can be achieved by multiple reaction monitoring (MRM) or parallel reaction monitoring (PRM) targeted proteomics approaches with suitable internal standards.^{6, 13} However, the existing proteomics approaches have two main analytical challenges for root cause investigation of PS-80 degradation.

First, the detection limit of current proteomics strategies depends on HCP abundances. Most reported proteomics-based approaches have detection limits at the sub-ppm level (HCP compared to drug substance, i.e. 6 orders of magnitude difference), which is limited by the dynamic range of the mass spectrometer. Some lipases, such as LPLA2, can significantly degrade polysorbate even below sub-ppm level,⁶ which is around the threshold or beyond most proteomics platforms. Thus research efforts have been focused on reducing the samples dynamic range to improve assay sensitivity, such as limited native digestion¹⁷⁻¹⁸, molecular weight cut-off²⁰ or 2D-LC^{17, 19}.

Second, enzyme abundance cannot tell the whole story of polysorbate degradation. Different lipases/esterases may have different enzyme activity and impact on polysorbate degradation. Determining the enzyme activity is key in understanding the root-cause of the degradation. There is huge analytical gap between enzyme abundance and the correlated activity for polysorbate degradation in biotherapeutics process and formulation development.

Serine hydrolases are one of the largest known enzyme classes, comprising approximately 200 enzymes or 1% of the genes in the human proteome.²¹⁻²² Included in this enzyme superfamily are multiple lipases, esterases, thioesterase, amidases and peptidases,²² which are all potentially harmful for biotherapeutics drug substances and formulation excipients. The defining characteristic of serine hydrolase superfamily members is the presence of a nucleophilic serine in the active site. This site can be covalently labelled with chemical probes for enrichment and subsequently identified by mass spectrometry.²¹⁻²⁴ This approach has been used to identify active serine hydrolases and screen inhibitors for serine hydrolases.²⁵⁻²⁸ There is a need to explore and improve the application of this activity-based chemical proteomics approach for active enzyme profiling to investigate polysorbate degradation in biotherapeutic formulations.

### SUMMARY OF THE INVENTION

The present disclosure describes a method of identifying serine hydrolase in a biological test sample obtained from protein production with a chemical probe such as a fluorophosphonate-containing probe. The present disclosure also provides a method of identifying one or more serine hydrolases in the biological test sample as causing PS-80 or PS-20 degradation. Compared with abundance-based host cell protein analytical assays, the present invention provides significant enrichment and detection of most host cell serine hydrolases including lipases.

### BRIEF DESCRIPTION OF THE DRAWINGS

FIG. 1. Evaluation of chemical probes for activity-based proteomics approach. Two positive probes (FP-Desthiobiotin and FP-Biotin) were evaluated using another probe with different enrichment group and DMSO as controls. Several enriched hydrolases which function as lipases and esterases are listed. The MS1 peak area was used for semi-quantification comparison.

FIG. 2. Assessment of assay reproducibility of the activity-based proteomics approach. Two experiments using the activity-based proteomics approach was performed independently with the HCCF sample from mAb1

FIG. 3A-3B. Spike-in UPS-2 to determine the sensitivity and semi-quantification of the traditional abundance-based proteomics approach. (3A) Spike-in proteins above 1 ppm were consistently identified with >2 unique peptides. (3B) The average MS signal from MS1 peak area correlated very well with the spiked UPS-2 protein concentration in general. The MS response varies among proteins from the same concentration range.

FIG. 4. The comparison of the serine hydrolase profile in HCCF from two mAbs using the activity-based proteomics approach. The MS1 peak area was used for semi-quantification comparison

### DETAILED DESCRIPTION OF THE INVENTION

The invention provides a method of identifying serine hydrolase in a biological test sample obtained from recombinant protein production, comprising the steps of
a. adding a fluorophosphonate probe to the biological test sample comprising a serine hydrolase in conditions that allow the probe to bind to the serine hydrolase;
b. removing the free fluorophosphonate probes not bound to the serine hydrolase;
c. enriching the probe-bound serine hydrolases with an agent that binds to the probes;
d. digesting the serine hydrolase; and
e. identifying the serine hydrolase in the test sample with mass spectroscopy.

In one embodiment, the agent in step c) has high affinity for an unbound moiety on the fluorophosphonate probe so as to enrich the probe-bound serine hydrolases. Examples include but are not limited to strepavidin or avidin beads. Then, the probe-bound serine hydrolase can undergo enzymatic digestion through for example, tyrpsin or chymotrypsin digestion. In one embodiment, the identified serine hydrolase has a 4-fold enrichment compared to the sample incubated with DMSO.

### I. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Flurophosphonate probe” is a compound comprising the moiety

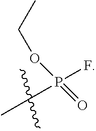 [SMILES: *P(=O)(F)OCC]

As used herein, “alkyl” is intended to include both branched and straight-chain saturated aliphatic hydrocarbon groups having the specified number of carbon atoms. For example, C1-C10, as in “C1-C10 alkyl” is defined to include groups having 1, 2, 3, 4, 5, 6, 7, 8, 9 or 10 carbons in a linear or branched arrangement. For example, “C1-C10 alkyl” specifically includes methyl, ethyl, n-propyl, i-propyl, n-butyl, t-butyl, i-butyl, pentyl, hexyl, heptyl, octyl, nonyl, decyl, and so on.

When used in the phrases “alkylheterocyclyl” the term “alkyl” refers to the alkyl portion of the moiety and does not describe the number of atoms in the heterocyclyl portion of the moiety. In an embodiment, if the number of carbon atoms is not specified, the “alkyl” of “alkylheterocyclyl” refers to C1-C20 alkyl and in a further embodiment, refers to C1-C15 alkyl.

“Heterocyclyl” means a non-aromatic saturated monocyclic, bicyclic, tricyclic or spirocyclic ring system comprising up to 7 atoms in each ring, or contains 3 to 14, or 5 to 10 ring atoms, in which one or more of the atoms in the ring system is an element other than carbon, for example, nitrogen, oxygen, phosphor or sulfur, alone or in combination. There are no adjacent oxygen and/or sulfur atoms present in the ring system. Preferred heterocyclyls contain about 5 to about 6 ring atoms. The heterocycle may be fused with an aromatic aryl group such as phenyl. The heterocyclyl is optionally bridged (i.e., forming a bicyclic moiety), for example with a methylene, ethylene or propylene bridge. The prefix aza-, oxa- or thia- before the heterocyclyl root name means that at least a nitrogen, oxygen or sulfur atom, respectively, is present as a ring atom. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” also includes heterocyclyl rings as described above wherein ═O replaces two available hydrogens on the same ring carbon atom.

### Serine Hydrolases

The serine hydrolase superfamily members include but are not limited to lipases, esterases, thioesterase, amidases and peptidases. Serine hydrolase include but are not limited to Pla2g7: phospholipase A2, group VII, LPL: lipoprotein lipase; Pla2g15 (LPLA2): phospholipase A2, group XV; Lypla2: lysophospholipase 2; Lyplal1 lysophospholipase-like 1; Plbd2: phospholipase B domain containing 2, Ppt2: palmitoyl-protein thioesterase 2, Ppme1: protein phosphatase methylesterase 1; Ces1g: carboxylesterase 1G; Uchl5: ubiquitin carboxyl-terminal esterase L5; Siae: sialic acid acetylesterase; Ces1gn: carboxylesterase 1G; acyl-coenzyme A thioesterase 1; Iah1: isoamyl acetate-hydrolyzing esterase 1; liver carboxylesterase 1-like protein; Ptgr2: prostaglandin reductase 2; Ctsa: cathepsin A; Pafah2: platelet-activating factor acetylhydrolase 2; Prep: prolyl endopeptidase; Rbbp9: retinoblastoma binding protein 9; Clrl: complement component 1; Apeh: acylpeptide hydrolase; Htra1: HtrA serine peptidase 1; Prcp: prolylcarboxypeptidase; Psmb5: proteasome subunit, beta type 5; Abhd4: abhydrolase domain containing 4; and Pafah1b3: platelet-activating factor acetylhydrolase, isoform 1b, subunit 3.

In certain embodiments, the serine hydrolase is a lipase selected from the group consisting of LPL or LPLA2 In yet another embodiment, the lipase is LPLA2. In other embodiments, the serine hydrolase is a lipase selected from the group consisting of LPLA2, PLA2G7 (phospholipase A2, group VII), PLA1A (phospholipase A1 member A), LYPLA2 (lysophospholipase 2) and LYPLAl1 (lysophospholipase-like 1). In one embodiment, the serine hydrolase is selected from the group consisting SIAE (sialic acid acetylesterase), CES1 (liver carboxylesterase 1), PREP (prolyl endopeptidase), PLA2G7 (phospholipase A2, group VII) and PRCP (prolylcarboxypeptidase). In another embodiment, the serine hydrolase is selected from the group consisting SIAE (sialic acid acetylesterase), CES1 (liver carboxylesterase 1), PREP (prolyl endopeptidase), PLA2G7 (phospholipase A2, group VII), PRCP (prolylcarboxypeptidase), LPLA2, PLA1A (phospholipase A1 member A), LYPLA2 (lysophospholipase 2) and LYPLAl1 (lysophospholipase-like 1).

The host cells producing the protein and serine hydrolase can be any cell used for expressing an exogenous protein. Common host cells used in manufacturing of biopharmaceuticals include but are not limited to CHO cells, baby hamster kidney (BHK21) cells, murine myeloma NS0 cells, murine myeloma Sp2/0 cells, human embryonic kidney 293 (HEK293) cells, fibrosarcoma HT-1080 cells, PER.C6 cells, HKB-11 cells, CAP cells, HuH-7 cells, murine C127 cells, and naturally generated or genetically modified variants thereof. In certain embodiments, the host cells are CHO cells. In some embodiments, the host cells are baby hamster kidney (BHK21) cells. In other embodiments, the host cells are murine myeloma NS0 cells. In yet other embodiments, the host cells are murine myeloma Sp2/0 cells. In still other embodiments, the host cells are human embryonic kidney 293 (HEK293) cells. In certain embodiments, the host cells are fibrosarcoma HT-1080 cells. In some embodiments, the host cells are PER.C6 cells. In other embodiments, the host cells are HKB-11 cells. In yet other embodiments, the host cells are CAP cells. In still other embodiments, the host cells are HuH-7 cells. In certain embodiments, the host cells are murine C127 cells. In some embodiments, the host cells are a naturally generated variant of the above host cells. In other embodiments, the host cells are a genetically modified variant of the above host cells.

### Flurophosphonate Probe

Chemical probes that bind to serine hydrolase can be used in the present invention. Examples of chemical probes are disclosed in Chembiochem 2019, 20 (17), 2212-2216; and Long, J. Z. and Cravatt, B. F., Chem Rev 2011, 111 (10), 6022-63.

Serine hydrolases have a nucleophilic serine in the active site, which can be labelled with flurophosphonate chemical probes for imaging of activity and also for identification. In one embodiment, the flurophosphonate probe is

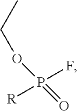

wherein R is alkyl, alkylheterocyclyl, —(CH₂)n₁-C(O)NH—(CH₂)n₂-NHC(O)(CH₂)n₃-R₁, —(CH₂)n₄-NHC(O)(CH₂)n₃-R₁, wherein n₁ is 8, 9, 10 or 11, n₂ is 2, 3, 4 or 5, n₃ is 2, 3, 4 or 5, n₄ is 8, 9, 10 or 11; and R₁ is heterocyclyl. In one embodiment, R₁ is

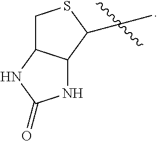 [SMILES: *C1SCC2NC(=O)NC21]

In one embodiment, R₁ is

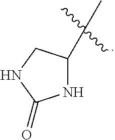 [SMILES: *C1CNC(=O)N1]

In one embodiment, n₁+n₂+n₃=16, 17, 18 or 19.

In another embodiment, the flurophosphonate probe is

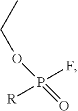

wherein R is —(CH₂)n₁-C(O)NH—(CH₂)n₂-NHC(O)(CH₂)n₃-R₁, wherein n₁ is 8, 9, 10 or 11, n₂ is 2, 3, 4 or 5, n₃ is 2, 3, 4 or 5, and R₁ is heterocyclyl. In one embodiment, R₁ is

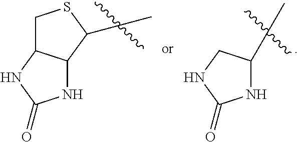 [SMILES: *C1SCC2NC(=O)NC21 ; *C1CNC(=O)N1]

In one embodiment, n₁+n₂+n₃=16, 17, 18 or 19. In one embodiment, n₁+n₂+n₃=18. In one embodiment, the flurophosphonate probe is

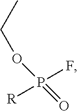

wherein R is —(CH₂)n₁-C(O)NH—(CH₂)n₂-NHC(O)(CH₂)n₃-R₁, wherein n₁ is 9, n₂ is 5, n₃ is 4, and R₁ is heterocyclyl. In one embodiment, R₁ is

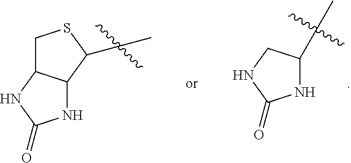 [SMILES: *C1SCC2NC(=O)NC21 ; *C1CNC(=O)N1]

In one embodiment, the flurophosphonate probe is

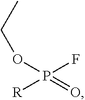

wherein R is —(CH₂)n₄-NHC(O)(CH₂)n₃-R₁, wherein n₃ is 5, n₄ is 10, and R₁ is heterocyclyl. In one embodiment, R₁ is

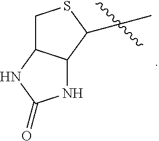 [SMILES: *C1SCC2NC(=O)NC21]

In one embodiment, R₁ is

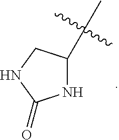 [SMILES: *C1CNC(=O)N1]

In one embodiment, the flurophosphonate probe

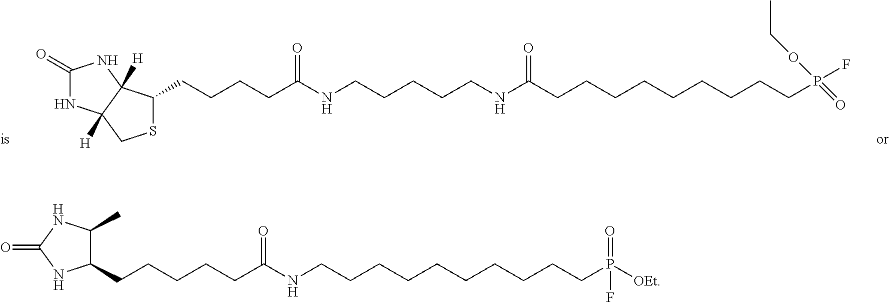 [SMILES: CCOP(=O)(F)CCCCCCCCCC(=O)NCCCCCNC(=O)CCCC[C@@H]1SC[C@@H]2NC(=O)N[C@@H]21 ; CCOP(=O)(F)CCCCCCCCCCNC(=O)CCCCC[C@H]1NC(=O)N[C@H]1C]

### Biological Test Sample

The biological test sample can be a harvest cell culture fluid (HCCF), including a HCCF sample that has undergone a chromatographic process for the separation of host cell serine hydrolase from the protein of interest. In one embodiment, the protein of interest is a monoclonal antibody. The chromatographic process may include but is not limited to one or more of a CEX, AEX, mixed mode IEX, mixed mode AEX, mixed mode CEX, affinity chromatographic process such as protein A or protein G affinity chromatographic process, immobilized metal affinity chromatographic (IMAC) process, HAC and HIC chromatographic process.

IEX chromatography separates molecules based on the net charge of the molecules. Separation occurs as a result of competition between the charged molecule of interest and counter ions for oppositely charged ligand groups on the IEX chromatographic resin. Strength of the binding of the molecule to the IEX resin depends on the net charge of the molecules, which is affected by operating conditions, such as pH and ionic strength. IEX resins include AEX resins and CEX resins. AEX resins may contain substituents such as diethylaminoethyl (DEAE), trimethyalaminoethyl (TMAE), quaternary aminoethyl (QAE) and quaternary amine (O) groups. CEX resins may contain substituents such as carboxymethyl (CM), sulfoethyl (SE), sulfopropyl (SP), phosphate (P) and sulfonate (S). Cellulosic IEX resins such as DE23, DE32, DE52, CM-23, CM-32 and CM-52 are available from Whatman Ltd. Maidstone, Kent, U.K. Sephadex-based and cross-linked IEX resins are also known. For example, DEAE-, QAE-, CM-, and SP-Sephadex, and DEAE-, Q-, CM- and S-Sepharose, and Sepharose are all available from GE Healthcare, Piscataway, N.J. Further, both DEAE and CM derived ethylene glycol-methacrylate copolymer such as TOYOPEARL™ DEAE-650S or M and TOYOPEARL™ CM-650S or M are available from Toso Haas Co., Philadelphia, Pa. POROS™ HS, POROS™ HQ, POROS™ XS are available from Thermo Fisher Scientific, Waltham, Mass.

HIC chromatography separates molecules based on the hydrophobicity of molecules. Hydrophobic regions in the molecule of interest bind to the HIC resin through hydrophobic interaction. Strength of the interaction depends on operating conditions such as pH, ionic strength, and salt concentration. In general, HIC resins contain a base matrix (e.g., cross-linked agarose or synthetic copolymer material) to which hydrophobic ligands (e.g., alkyl or aryl groups) are coupled. Non-limiting examples of HIC resins include Phenyl SEPHAROSE™ 6 FAST FLOW™ (Pharmacia LKB Biotechnology, AB, Sweden); Phenyl SEPHAROSE™ High Performance (Pharmacia LKB Biotechnology, AB, Sweden); Octyl SEPHAROSE™ High Performance (Pharmacia LKB Biotechnology, AB, Sweden); Fractogel™ EMD Propyl or FRACTOGEL™ EMD Phenyl (E. Merck, Germany); MACRO-PREP™ Methyl or MACRO-PREP™ t-Butyl Supports (Bio-Rad, CA); WP HI-Propyl (C3)™ (J. T. Baker, NJ); TOYOPEARL™ ether, phenyl or butyl (TosoHaas, Pa.); and Tosoh-Butyl-650M (Tosoh Corp., Tokyo, Japan).

HAC chromatography uses an insoluble hydroxylated calcium phosphate of the formula [Ca₁₀(PO₄)₆(OH)₂] as both the matrix and the ligand. The functional groups of the HAC resin include pairs of positively charged calcium ions (C-sites) and negatively charged phosphate groups (P-sites). The C-sites can interact with carboxylate residues on the protein surface while the P-sites can interact with basic protein residues. Strength of the binding between the protein and the HAC resin depends on operating conditions including pH, ionic strength, composition of solution, concentration of each component of the composition, gradient of pH, gradient of component concentration, etc. Various HAC resins, such as CHT™ Ceramic Hydroxyapatite and CFT™ Ceramic Fluoroapatite, are commercially available.

Affinity chromatography separates molecules based on a highly specific interaction between the molecule of interest and the functional group of the resin, such as interaction between antigen and antibody, enzyme and substrate, receptor and ligand, or protein and nucleic acid, etc. Some commonly used affinity chromatographic resins include protein A or protein G resin to purify antibodies, avidin biotin resin to purify biotin/avidin and their derivatives, glutathione resin to purify GST-tagged recombinant proteins, heparin resin to separate plasma coagulation proteins, IMAC resin to purify proteins that specifically interact with the metal ions, etc. Operating conditions of each affinity chromatography depend on the mechanism of the interaction and factors that affect the interaction. Commercial affinity chromatographic resins include but are not limited to MabSelect Sure, UNOsphere SUPrA™, Affi-Gel®, and Affi-Prep®.

Mixed mode chromatographic processes can be a combination of any two or more functions or mechanisms described above or understood by a person of ordinary skill in the art, such as a combination of IEX and HIC (e.g., AEX/HIC or CEX/HIC), a combination of AEX and CEX (AEX/CEX), a combination of HIC, AEX, and CEX (HIC/AEX/CEX), etc. Exemplary mixed mode chromatographic resins include but are not limited to OminPac PCX-500, Primesep®, Obelisc R, Oblisc N, Acclaim Trinity P1, Acclaim Trinity P2, Capto Adhere, Capto Adhere Impres, Capto MMC, Capto MMC Impres, Capto Core 700, PPA Hypercel, HEA Hypercel, MEP Hypercel, Eshmuno HCX, Toyopearl MX-Trp-650M, Nuvia C Prime, CHT Type I, and CHT Type II.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J Immunol. 165:6205; He, et al. (1998) J Immunol. 160:1029; Tang et al. (1999) J. Biol. Chem. 274:27371-27378; Baca et al. (1997) J Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fuse with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J Immunol. 163:5157-5164).

### EXAMPLES

Dithiothreitol (DTT) and high capacity streptavidin agarose were purchased from Pierce (Rockford, Ill.). Tris-HCl buffer and high-performance liquid chromatography (HPLC) grade solvents, DMSO, 10% SDS solution, acetonitrile (ACN) and Formic acid (FA) were purchased from Thermo Fisher Scientific (Waltham, Mass.). Monoclonal antibodies (mAbs) were obtained from Merck pipeline under preclinical or clinical development. Universal Proteomics Standard 2 (UPS2) and Urea were purchased from Sigma (Waltham, Mass.) Trypsin was purchased from Promega (Madison, Wis.). ActivX® Desthiobiotin-FP probe

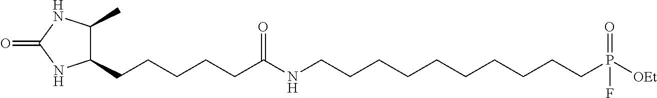 [SMILES: CCOP(=O)(F)CCCCCCCCCCNC(=O)CCCCC[C@H]1NC(=O)N[C@H]1C]

was from Thermo Fisher Scientific (Waltham, Mass.). FP-Biotin (P51) probe

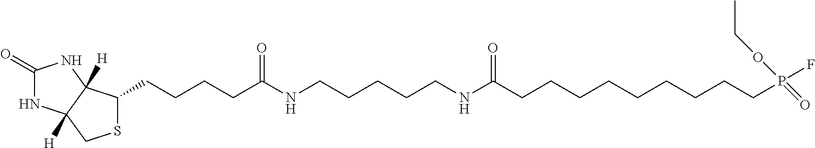 [SMILES: CCOP(=O)(F)CCCCCCCCCC(=O)NCCCCCNC(=O)CCCC[C@@H]1SC[C@@H]2NC(=O)N[C@@H]21]

and negative control probe P35

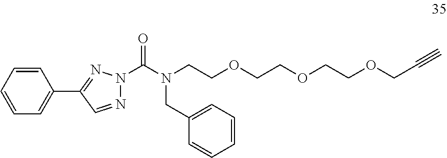 [SMILES: C#CCOCCOCCOCCN(Cc1ccccc1)C(=O)n1ncc(-c2ccccc2)n1]

were obtained from Merck Chemical Biology Department.

### Example 1: Activity-Based Chemical Proteomics Approach

Harvest cell culture fluid (HCCF) and products from the first ion-exchange column (IEXP)) in downstream purification were used for activity-based proteomics testing. HCCF from Chinese hamster ovary (CHO) cells from fed-batch production of mAbs was diluted with 50 mM Tris (pH 8) to 2 mg/mL. IEXP samples were diluted to 10 mg/mL. Fluorophosphonate (FP)-containing probes were dissolved in dirnethyl sulfoxide (DMSO) to make 0.1 mM stock solution. For each HCCF or IEXP sample (500 μL), 20 μL of chemical probes or control DMSO was added to make a final mix concentration of 2 μM. All samples were incubated at room temperature for 2 hours with constant mixing on a rotator. After reaction, to each sample was added 1000 μL of ice-cold methanol/acetone (50:50) on ice for 30 minutes to remove free probes. The precipitated proteins were collected via centrifugation at 20,000 g for 15 minutes at 4° C. The pellet was washed with 1 mL of ice-cold methanol/acetone solution once and dried under N₂ gas. The remaining pellet was re-dissolved in 1 ml of sample suspension buffer (2M Urea, 1% SDS, 50 mM Tris, pH 8). For each sample, 60 μL of Pierce™ agarose beads were added before incubation at room temperature for 1 hour while constantly mixing on a rotator. The avidin agarose beads were pelleted by centrifugation at 1000×g for 1 minute at room temperature and washed with 500 μL of 2 M Urea/50 mM Tris 3 times and 50 mM Tris 2 times. The washed agarose beads were resuspended in 50 μL of 50 mM Tris-HCl. Trypsin (2 μg) was added and digested at 37° C. on a thermomixer at 500 rpm overnight. The supernatant was collected after centrifugation at 10,000×g for 5 mins at room temperature. The collected samples were reduced at 80° C. for 10 mins with 1 μl DTT (1 M), and subsequently had 2 μL of 20% formic acid added before LC-MS/MS analysis.

Considering the low levels of peptides harvested from the activity-based chemical proteomics approach, the LC-MS was run with nanoflow on an EASY-nLC 1200 System (Thermo., Bremen, Germany), which was coupled with a Q Exactive™ HF-X Hybrid Quadrupole-Orbitrap™ mass spectrometry (Thermo, Bremen, Germany). EASY-Spray C18 column was used (PepMap RSLC, ES803, 2 μm, 100 Å, 75 μm×50 cm) with flow rate at 250 nl/min and column temperature at 40° C. Mobile phase A was made of MS grade water with 0.1% FA, and Mobile phase B was made of MS grade 80% ACN with 0.1% FA. The gradient started with 2% B for 3 mins, and changed to 45% at 83 min, and increased to 70% at 98 mins. The column was washed with 100% B from 100 mins to 110 mins, followed by 2 cycles of zig-zag washing step from 2% B to 100% B to reduce carry-over peptides. The MS was run with data dependent analysis (DDA) with MS1 scan range from 350 to 2000 m/z and top 10 most abundant ions for MS/MS fragmentation. The MS1 resolution was 60,000, AGC target 1 e⁶ and maximum IT for 60 ms. The MS2 resolution was 15,000, AGC target 1^{e} and maximum IT for 100 ms, isolation window at 1.4 m/z and NCE at 28. The dynamic exclusion was set for 10s. The ESI source was run with sheath gas flow rate at 8, aux gas flow rate at 0, spray voltage at 3.8 kV, capillary temp at 320° C., and Funnel RF level at 50.

### Example 2: Abundance-Based Traditional Proteomics Approach

For abundance-based proteomics approach, the sample preparation followed the native digestion method¹⁸. Briefly, one mg of mAb process intermediate samples from purification steps were incubated with trypsin (400:1, weight to weight) after pH adjust with 1M Tris-HCL for overnight digestion at 37° C. To determine the limit of detection of the traditional proteomics workflow, UPS2, composed of 48 human proteins (6,000 to 83,000 Da) within a concentration range from 250 amol to 25 pmol with 8 proteins in each group, was spiked in a mAb drug substance before sample preparation. After digestion, samples were denatured and reduced at 80° C. for 10 min with 2 μL of 50 mg/mL DTT. A large portion of undigested mAb was removed by centrifugation at 11,000 g for 10 min. Three microliters of 20% FA were added to the supernatant before LC-MS analysis.

LC-MS was performed on an ACQUITY UPLC H-Class system (Waters, Milford, Mass.) coupled with a Q Exactive™ HF-X Hybrid Quadrupole-Orbitrap™ mass spectrometer (Thermo, Bremen, Germany). The columnn was ACQUITY UPLC Peptide CSH C18 column (130 Å, 1.7 μm, 1×150 mm, Waters, Milford, Mass.) with flow rate at 50 μl/min and column temperature at 50° C. Mobile phase A was MS grade water with 0.1% FA, and Mobile phase B was MS grade ACN with 0.1% FA. The gradient started with 1% B for 5 min, and changed to 5% at 6 min, and increased to 26% at 85 min. The column was washed with 90% B from 90 mins to 105 mins, followed by 2 cycles of zig-zag washing step from 5% B to 90% B to reduce carry-over peptides. The MS was run with DDA with MS1 scan range from 300 to 1800 m/z and top 20 for MS/MS fragmentation. The MS1 resolution was 60,000, AGC target 1e⁶ and maximum IT for 60 ins. The MS2 resolution was 15,000, AGC target 1e⁵ and maximum IT for 100 ms, isolation window at 1.4 m/z and NCE at 27. The dynamic exclusion was set for 20s. The ESI source was run with sheath gas flow rate at 35, aux gas flow rate at 10, spray voltage at 3.8 kV, capillary temp at 275° C., Funnel RF level at 35 and aux gas heater temp at 100° C.

### Example 3: Proteomics Identification

MS raw data was searched against Merck internal CHO KL fasta database customized with mAb and spiked-in recombinant protein sequences using Proteome Discoverer 2.2. The precursor mass tolerance was set at 15 ppm and fragment mass tolerance at 0.02 Da. The dynamic modification was set for Met oxidation and maximum 3 modification. The target FDR for peptide identification was 0.01 and protein identification filter required at least 2 peptide identification. The summed MS1 peak area from all identified peptides were used for protein relative abundance estimation. In the activity-based proteomics approach, proteins with at least 4-fold enrichment compared to those from samples incubated with only DMSO were considered potential active serine hydrolases.

### Method Development and Evaluation of Activity-Based Chemical Proteomics Approach for Active Serine Hydrolases Profiling in Biologics Cell Culture

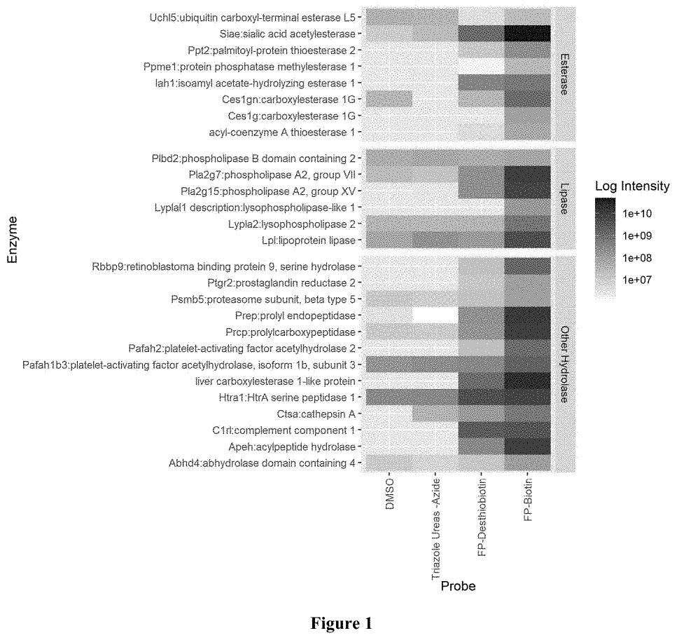

To establish the activity-based chemical proteomics approach, two commercial chemical probes were evaluated using an HCCF sample from mAb1. The enrichment of serine hydrolases, mostly lipases, was used for consideration compared with DMSO or other chemical probe with an orthogonal enrichment tag. As shown in FIG. 1, the FP-Biotin probe shows stronger enrichment for serine hydrolases compared with the FP-Desthiobiotin probe. More than 10 known lipases and esterases combined were significantly enriched with the FP-biotin probe. Some of those lipases have been reported to impact PS degradation, such as LPLA2 (Pla2g15, phospholipase A2, group XV)⁶ and LPL (lipoprotein lipase).⁵ One of the most problematic lipases from biologics purification, Plbl2 (Plbd2, phospholipase B domain containing 2),¹¹ has limited enrichment compared to other lipases. Based on the enrichment performance of known serine hydrolases, FP-Biotin was chosen for the rest of activity-based proteomics analysis.

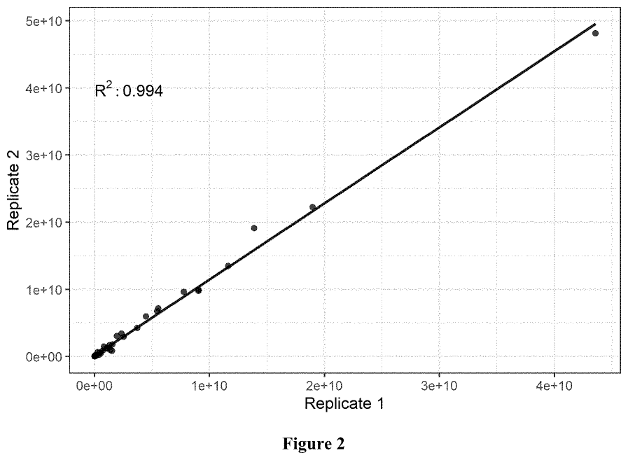

To evaluate assay reproducibility of the activity-based chemical proteomics approach, an HCCF sample from mAb1 was tested with two independent experiments. As shown in FIG. 2, comparison of the peak areas for commonly enriched proteins between the two preparations demonstrates great assay reproducibility (R²>0.99).

### Advantages of Activity-Based Proteomics Compared to Traditional Proteomics

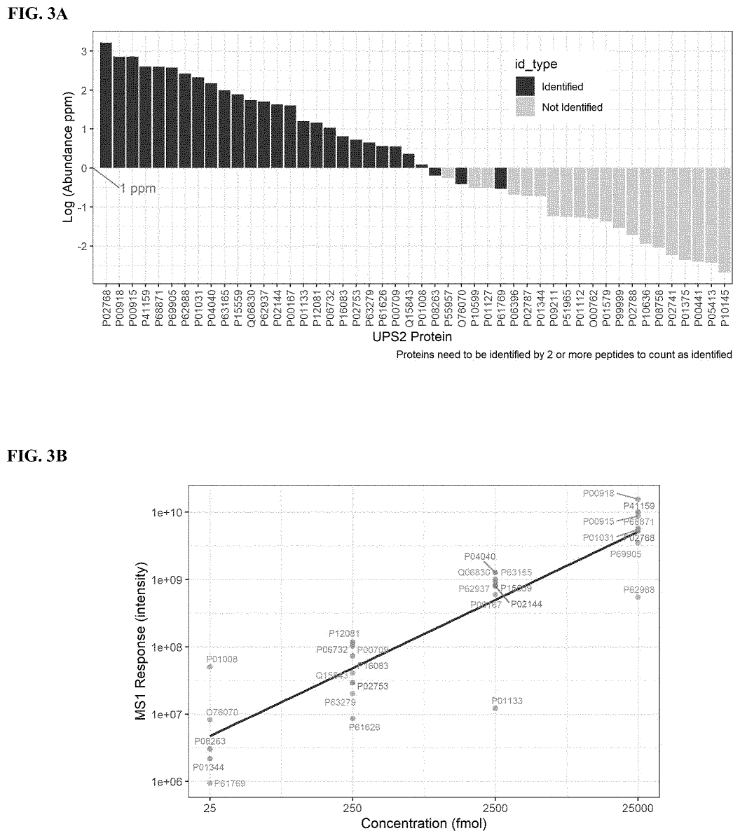

Keeping in mind the strengths of the ELISA assay for total HCP quantitation, we have positioned the LC-MS based proteomics approach as an orthogonal assay for specific HCP characterization during process development. There is no major bias of HCP identification for this shotgun-based proteomics approach except HCP abundance. To evaluate the detection limit of the established traditional proteomics approach, a UPS2 standard was spiked in a mAb DS before sample preparation and LC-MS analysis. All HCPs were confidently identified more than with 0.6 ppm and at least unique peptides (Table 1 and FIG. 3A). The average MS peak area extracted by Proteome Discoverer 2.2 software correlates well with the abundance of the spike-in protein (Table 1 and FIG. 3B).

TABLE 1
Evaluation of the detection limit of the abundance-
based proteomics approach using UPS2

| UPS2 | Range | ID (≥2 unique | Average MS1 |
|---|---|---|---|
| (fmol) | (ppm) | peptides) | peak area |
| 25,000 | 213-1659 | 8/8 | 6.82E+09 |
| 2500 | 16-149 | 8/8 | 7.98E+08 |
| 250 | 2-15 | 8/8 | 6.36E+07 |
| 25 | 0.2-1.2 | 5/8 | 1.29E+07 |

TABLE 2
The detected MS signal (MS1 peak area) from activity-based chemical
proteomics and abundance-based proteomics from mAb1 and mAb2

| | mAb1 | mAb1 | mAb2 | mAb2 |
|---|---|---|---|---|
| Description | (Activity) | (Abundance) | (Activity) | (Abundance) |
| Pla1a: phospholipase A1 | ND | ND | 3.26E+08 | ND |
| member A | | | | |
| Pla2g15: phospholipase A2, | 5.51E+09 | 3.65E+07 | 4.73E+09 | ND |
| group XV | | | | |
| Pla2g7: phospholipase A2, | 9.57E+09 | 2.57E+07 | 8.91E+09 | 1.83E+07 |
| group VII | | | | |
| Lypla2: lysophospholipase 2 | 1.45E+09 | ND | 6.58E+08 | ND |
| Lpl: lipoprotein lipase | 3.57E+09 | 3.54E+09 | 3.83E+08 | 8.74E+08 |
| Lyplal1: lysophospholipase- | 1.32E+08 | ND | 1.49E+08 | ND |
| like 1 | | | | |
| Lipa: lysosomal acid lipase A | ND | 1.54E+08 | 2.19E+08 | 4.45E+08 |
| Plbd2: phospholipase B | 5.26E+07 | 2.16E+09 | 2.27E+07 | 1.54E+08 |
| domain containing 2 | | | | |

ND: not detected

To assess the advantages of the activity-based proteomics approach compared to the abundance-based proteomics approach for low abundance HCP identification, the MS peak area of all the identified lipases from the two proteomic approaches in two mAbs (mAb1 and mAb2) were compared. As shown in Table 2, the activity-based proteomics approach resulted in more significant enrichment for most lipases compared to the abundance-based proteomics approach. The increase of MS signal may be from much reduced sample matrix and ion suppression from drug substance. For example, Lpla2 has been demonstrated to have significant impact on PS-80 degradation even at concentrations below 1 ppm.⁶ The MS signal of Lpla2 (pla2g15) from the activity-based approach was increased more than 150-fold compared with that from abundance-based proteomics. Pla2g7, a lipase from the same family as Lpla2, achieved even greater enrichment (>350) compared with Lpla2. Some lipases were only identified from the activity-based proteomics approach, such as Pla1a (phospholipase A1 member A), Lypla2 (lysophospholipase 2) and Lyplal1 (lysophospholipase-like 1). Several enzymes are detected with very high abundances by the activity proteomics approach, which include SIAE (sialic acid acetylesterase), CES1 (liver carboxylesterase 1), PREP (prolyl endopeptidase), PLA2G7 (phospholipase A2, group VII) and PRCP (prolylcarboxypeptidase). The high abundance of those active enzymes needs to be carefully controlled in biologics process development as they may have adverse impact on the stability of drug substance or formulation excipients. Interestingly, some lipases were found to have higher signal or only detected from the abundance-based proteomics, such as Lipa (lysosomal acid lipase A), Lpl (lipoprotein lipase) and Plbd2 (phospholipase B domain containing 2). This may suggest those lipases have less activity towards the chemical probes used in this study.

Early study suggested PLBL2 to be potentially the root cause of PS-20 degradation in a sulfatase drug product,⁴ however, the amount of PLBL2 (90% pure from a non-CHO species) used for functional confirmation was much higher than the concentration observed in drug product sample. 4 No lipase activity of PLBL2 is consistent with early report that purified recombinant CHO PLBL2 or mAb biotherapeutic in-process samples showed no in vitro phospholipase activity against synthetic substrates.¹⁴ Actually, phospholipase B-like protein from bovine was proposed to be an amidase or peptidase instead of a lipase, and a natural substrate for any members of the PLBD family has not been reported.³⁹ Our data suggests that CHO PLBL2 is most likely not an amidase or peptidase in the serine hydrolase family as it is not enriched by the active chemical probes or it is present in the sample in an inactive conformation. Overall, the impact of PLBL2 on PS degradation at concentrations detected in biotherapeutics is questionable, although the level of PLBL2 needs to be controlled because of immunogenicity risk.¹⁴ We have observed protein formulations containing over 500 ppm of PLBL2 without any PS-80 degradation (unpublished results). The role of different pH, buffer conditions, post-transitional modifications and association with drug substance for PLBL2 on PS degradation needs further study.

Profiling Serine Hydrolase from Two mAbs

To understand the protein composition of enriched proteins by the activity-based proteomics approach, HCCF samples from two mAbs against different antigens from two different CHO cell lines were tested. Of all the 82 proteins enriched more than 4-fold compared to DMSO control from at least one mAb, 55 proteins (>67%) have functions annotated as hydrolases, and 27 of 82 proteins have unannotated functions as hydrolases. The proteins enriched by the activity-based proteomics approach also help define and annotate new serine hydrolases.

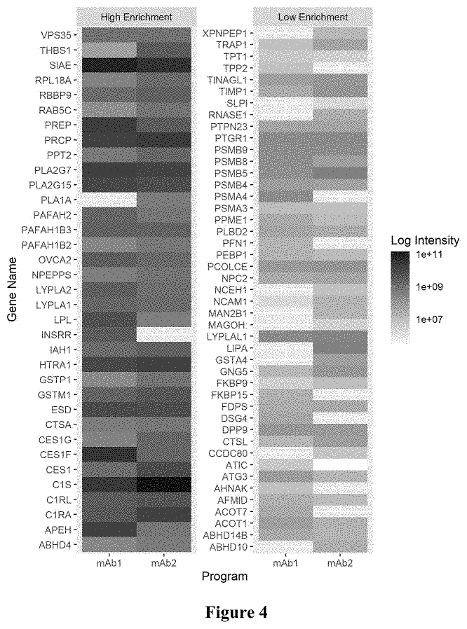

In order to assess the serine hydrolase profiling between different biotherapeutic molecules, the enriched known hydrolases from the activities-based proteomics were compared between mAb1 and mAb2. More than 36% of enriched hydrolases are only identified from one mAb or show more than 4-fold differences. The remaining ones are comparable between the two mAbs (FIG. 4).

Serine hydrolases are a large functionally diverse group of enzymes with great biological and pharmaceutical importance.²¹⁻²² By using a base-activated serine as a nucleophile, these enzymes hydrolyze ester, thioester and amide bonds in a variety of substrates including metabolites, lipids, peptides and proteins. For this reason, they play important roles in physiological and pathological systems.²¹⁻²² Several lipases, esterases, thioesterase, amidases and peptidases belong to serine hydrolases superfamily.²¹⁻²² Those serine hydrolases secreted from host cell lines or released from broken cells during cell culture have been reported to significantly impact biological drug substance and its corresponding formulation excipients. For example, complement component 1's (CIs), a serine protease from the complement cascade, was shown to be responsible for the proteolysis of protein gp120 in CHO cells²⁹. The protease from serine hydrolases, such as CIs and Cathepsin A, have hindered the development of biologics including HIV vaccines²⁹. Several lipases or esterases such as Lpla2, Lpl and Plbl2 have been reported to degrade the polysorbate formulation excipient.⁴⁻⁷ Problematic proteases and lipases from HCP are usually identified by abundance-based proteomics; the sensitivity of which is limited by the high abundance drug substance in a sample with extensive purification. Their functional consequences were demonstrated sometimes by spiking-in, not fully purified recombinant proteins with much higher concentrations than those detected from endogenous samples. On the other hand, not all proteases and lipases have the same activity at the same concentration. It is possible that the most active hydrolases can cause noticeable damage while being below the detection limit of the abundance-based proteomics methods. Hence, it is critical to develop a method that can selectively enrich those proteins in order to explain some of the adverse impact from HCP observed on drug substance or formulation excipient.

Based on the mechanism of action of serine hydrolases, activity-based protein profiling or activity-based proteomics approach has been developed to specifically enrich and identify proteins from this superfamily.^{21-22, 30} Covalent protein modifiers or chemical probes play a key role as starting points for designing irreversible enzyme inhibitors and developing chemical probes for activity-based protein profiling.³⁰ To optimize the activity-based proteomics approach for serine hydrolases profiling in HCCF from biologics cell culture, two commercially available FP probes were evaluated using the same workflow. As shown in FIG. 1, FP-Biotin probe has better enrichment for most serine hydrolases compared with FP-Desthiobiotin probe. It is possible different chemical probes have different affinity for various serine hydrolyses. There has been significant research focused on chemical probe development for selected groups of serine hydrolyses or for different applications.³¹⁻³² Theoretically, various chemical probes with different affinity groups but the same purification group may be used to widen the detection range of serine hydrolases.³³ Not limited to serine hydrolases, activity-based proteomics can be used to identify active enzymes from cysteine or threonine proteases, aspartyl or metalloproteases and glycosidase using similar workflow but using different chemical probes.^{24, 34} Those enzymes may have similar properties as some biotherapeutic proteins, or bind to biotherapeutics for co-purification. For example, Cathepsin D, a member of aspartyl protease family, caused antibody fragment or particle formation in monoclonal antibody products.³⁵⁻³⁶ While in another case, Cathepsin L caused proteolytic cleavage of CHO expressed proteins during processing and storage.³⁷ Unlike Cathepsin D, Cathepsin L belongs to the cysteine protease family. In all those studies, extensive enrichment was performed in order to identify those low abundance HCP enzymes.³⁵⁻³⁷ There is opportunity to develop a universal activity-based proteomics approach to identify a dozen members of Cathepsin family using a pool of chemical probes for active serine, cysteine and aspartyl proteases.

To the best of our knowledge, this study demonstrated the first application of activity-based proteomics for serine hydrolase profiling in cell culture fluid from cells used to produce therapeutic proteins. The composition and activity of serine hydrolases will help understand the source of those HCPs and their potential impact for biologics drug substance and its formulation excipients. The activity-based proteomics approach also helps identify and annotate proteins with potential new functions depending on the specific chemical probes used. The understanding of serine hydrolase profiling, especially those enzymes known to have adverse impact, such as proteases and lipases, will provide extremely useful information for cell line development, clone selection, upstream and downstream development. In our pilot study, the serine hydrolase composition of HCP from two mAbs show distinct profiling. The difference may be from different strains of CHO cell lines, the impact of biotherapeutic proteins and cell culture conditions. This provides a rationale to evaluate serine hydrolase composition for each therapeutic protein.

### Example 4: Lipase Activity Determination by PS-80 Force Degradation

The lipase/esterase activity from HCPs in process intermediates was determined using PS-80 incubation study. PS-80 (0.02%, w/v) was incubated with or without process intermediates at 37° C. for 14 days. The PS-80 concentration was determined by HPLC-CAD (Corona Charged Aerosol Detector, Thermo, Bremen, Germany) with Oasis Max column (2.1×20 mm, 30 μm, Waters, Milford, Mass.). Mobile phase A was 0.5% (v/v) acetic acid in water. Mobile phase B was 0.5% acetic acid in isopropyl alcohol. PS-80 was eluted with 20% to 70% mobile phase B from 3 to 11 mins. Standard curves with 25% to 150% of target concentration was run before and after testing samples to make standard curves for PS-80 concentration determination. A 10% PS-80 degradation was considered as action limit for potential HCP mitigation investigation.

To demonstrate the application of the activity-based proteomics approach to root cause investigation during bioprocess development, the IEXP samples after the first polish column from mAb2 was analyzed by both proteomics approaches. IEXP samples are purified by ProA and ion exchange columns from HCCF. Thus, IEXP samples contain much less HCPS than HCCF samples and those remaining HCPs in the IEXP are usually extremely low. PS-80 incubation study indicated there was significant degradation for mAb2 compared to the placebo control that doesn't contain drug substance (Table 3). The abundance-based proteomics failed to identify any lipase in mAb2. By contrast, in the activity-based proteomics, two serine hydrolases Pla2g7 (phospholipase A2, group VII) and Siae (sialic acid acetylesterase) were identified from mAb2.

TABLE 3
The lipase/esterase identification from activity-
based proteomics and abundance-based proteomics
and their impact on PS-80 degradation

| | | | % PS-80 degradation |
|---|---|---|---|
| Product | | | PS-80 force |
| after polish | LC-MS based Proteomics | | degradation 37° C./D 14 |
| column | Abundance | Activity | (>10%) |
| mAb 2 | No | Pla2g7, Siae | 27.27 |
| | lipaseidentified | | |

The major application of this activity-based proteomics assay will be to investigate polysorbate degradation in biologics formulation. There is an industry-wide challenge to identify trace levels of lipases/esterase for PS-80 degradation using the existing analytical toolbox. Those active enzymes usually fall below the limit of detection of traditional abundance-based proteomics in a drug substance.⁶ It adds extra challenge to assess their activity for polysorbate degradation by just identifying the protein itself. There is no direct high-throughput assay to assess lipase activity. For example, our current lipase activity assay runs up to 2 or 3 weeks by incubating PS-80 degradation at 37° C. The activity-based proteomics potentially addresses both challenges by identifying those low abundant enzymes based on their activities. As a proof-of-concept study, process intermediates from mAb2 after a second column were subjected to both traditional abundance-based proteomics and activity-based proteomics analysis. As shown in Table 3, there was significant PS-80 degradation observed in mAb2, but no lipase or esterases identified by abundance-based proteomics. The activity-based proteomics identified two lipase or esterase. Pla2g7, also known as platelet-activating factor (PAF) acetylhydrolase, modulates the action of PAF by hydrolyzing the sn-2 ester bond to yield the biologically inactive lyso-PAF.³⁹ A member of this phospholipase A2 family, Lpla2 (Pla2g15), has shown to degrade both PS-80 and PS-20 as low as 0.3 ppm.⁶ The other esterase identified from mAb2, Siae (sialic acid acetylesterase) belongs to the family of hydrolases, specifically those acting on carboxylic ester bonds, which is same ester bond in PS-80. This case study demonstrated the advantage and potential of the activity-based proteomics approach in contrast to abundance-based proteomics approach for root cause investigation of polysorbate degradation.

### REFERENCES

1. Dwivedi, M.; Blech, M.; Presser, I.; Garidel, P., Polysorbate degradation in biotherapeutic formulations: Identification and discussion of current root causes. Int J Pharm 2018, 552 (1-2), 422-436.
2. Kerwin, B. A., Polysorbates 20 and 80 used in the formulation of protein biotherapeutics: structure and degradation pathways. J Pharm Sci 2008, 97 (8), 2924-35.
3. Kishore, R. S.; Kiese, S.; Fischer, S.; Pappenberger, A.; Grauschopf, U.; Mahler, H. C., The degradation of polysorbates 20 and 80 and its potential impact on the stability of biotherapeutics. Pharm Res 2011, 28 (5), 1194-210.
4. Dixit, N.; Salamat-Miller, N.; Salinas, P. A.; Taylor, K. D.; Basu, S. K., Residual Host Cell Protein Promotes Polysorbate 20 Degradation in a Sulfatase Drug Product Leading to Free Fatty Acid Particles. J Pharm Sci 2016, 105 (5), 1657-1666.
5. Chiu, J.; Valente, K. N.; Levy, N. E.; Min, L.; Lenhoff, A. M.; Lee, K. H., Knockout of a difficult-to-remove CHO host cell protein, lipoprotein lipase, for improved polysorbate stability in monoclonal antibody formulations. Biotechnol Bioeng 2017, 114 (5), 1006-1015.
6. Hall, T.; Sandefur, S. L.; Frye, C. C.; Tuley, T. L.; Huang, L., Polysorbates 20 and 80 Degradation by Group XV Lysosomal Phospholipase A2 Isomer X1 in Monoclonal Antibody Formulations. J Pharm Sci 2016, 105 (5), 1633-1642.
7. McShan, A. C.; Kei, P.; Ji, J. A.; Kim, D. C.; Wang, Y. J., Hydrolysis of Polysorbate 20 and 80 by a Range of Carboxylester Hydrolases. PDA J Pharm Sci Technol 2016, 70 (4), 332-45.
8. Tomlinson, A.; Demeule, B.; Lin, B.; Yadav, S., Polysorbate 20 Degradation in Biopharmaceutical Formulations: Quantification of Free Fatty Acids, Characterization of Particulates, and Insights into the Degradation Mechanism. Mol Pharm 2015, 12 (11), 3805-15.
9. Cao, X.; Fesinmeyer, R. M.; Pierini, C. J.; Siska, C. C.; Litowski, J. R.; Brych, S.; Wen, Z. Q.; Kleemann, G. R., Free fatty acid particles in protein formulations, part 1: microspectroscopic identification. J Pharm Sci 2015, 104 (2), 433-46.
10. Labrenz, S. R., Ester hydrolysis of polysorbate 80 in mAb drug product: evidence in support of the hypothesized risk after the observation of visible particulate in mAb formulations.

J Pharm Sci 2014, 103 (8), 2268-77.
11. Tran, B.; Grosskopf, V.; Wang, X.; Yang, J.; Walker, D., Jr.; Yu, C.; McDonald, P., Investigating interactions between phospholipase B-Like 2 and antibodies during Protein A chromatography. J Chromatogr A 2016, 1438, 31-8.
12. Bracewell, D. G.; Francis, R.; Smales, C. M., The future of host cell protein (HCP) identification during process development and manufacturing linked to a risk-based management for their control. Biotechnol Bioeng 2015, 112 (9), 1727-37.
13. Gao, X.; Rawal, B.; Wang, Y.; Li, X.; Wylie, D.; Liu, Y. H.; Breunig, L.; Driscoll, D.; Wang, F.; Richardson, D. D., Targeted Host Cell Protein Quantification by LC-MRM Enables Biologics Processing and Product Characterization. Anal Chem 2019.
14. Fischer, S. K.; Cheu, M.; Peng, K.; Lowe, J.; Araujo, J.; Murray, E.; McClintock, D.; Matthews, J.; Siguenza, P.; Song, A., Specific Immune Response to Phospholipase B-Like 2 Protein, a Host Cell Impurity in Lebrikizumab Clinical Material. AAPS J2017, 19 (1), 254-263.
15. Vanderlaan, M.; Zhu-Shimoni, J.; Lin, S.; Gunawan, F.; Waemer, T.; Van Cott, K. E., Experience with host cell protein impurities in biopharmaceuticals. Biotechnol Prog 2018, 34 (4), 828-837.
16. Walker, D. E.; Yang, F.; Carver, J.; Joe, K.; Michels, D. A.; Yu, X. C., A modular and adaptive mass spectrometry-based platform for support of bioprocess development toward optimal host cell protein clearance. MAbs 2017, 9 (4), 654-663.
17. Kufer, R.; Haindl, M.; Wegele, H.; Wohlrab, S., Evaluation of Peptide Fractionation and Native Digestion as Two Novel Sample Preparation Workflows to Improve HCP Characterization by LC-MS/MS. Anal Chem 2019, 91 (15), 9716-9723.
18. Huang, L.; Wang, N.; Mitchell, C. E.; Brownlee, T.; Maple, S. R.; De Felippis, M. R., A Novel Sample Preparation for Shotgun Proteomics Characterization of HCPs in Antibodies. Anal Chem 2017, 89 (10), 5436-5444.
19. Yang, F.; Walker, D. E.; Schoenfelder, J.; Carver, J.; Zhang, A.; Li, D.; Harris, R.; Stults, J. T.; Yu, X. C.; Michels, D. A., A 2D LC-MS/MS Strategy for Reliable Detection of 10-ppm Level Residual Host Cell Proteins in Therapeutic Antibodies. Anal Chem 2018, 90 (22), 13365-13372.
20. Chen, I. H.; Xiao, H.; Daly, T.; Li, N., Improved Host Cell Protein Analysis in Monoclonal Antibody Products through Molecular Weight Cutoff Enrichment. Anal Chem 2020, 92 (5), 3751-3757.
21. Simon, G. M.; Cravatt, B. F., Activity-based proteomics of enzyme superfamilies: serine hydrolases as a case study. J Biol Chem 2010, 285 (15), 11051-5.
22. Long, J. Z.; Cravatt, B. F., The metabolic serine hydrolases and their functions in mammalian physiology and disease. Chem Rev 2011, 111 (10), 6022-63.
23. Adam, G. C.; Sorensen, E. J.; Cravatt, B. F., Chemical strategies for functional proteomics. Mol Cell Proteomics 2002, 1 (10), 781-90.
24. Sanman, L. E.; Bogyo, M., Activity-based profiling of proteases. Annu Rev Biochem 2014, 83, 249-73.
25. Zweerink, S.; Kallnik, V.; Ninck, S.; Nickel, S.; Verheyen, J.; Blum, M.; Wagner, A.; Feldmann, I.; Sickmann, A.; Albers, S. V.; Brasen, C.; Kaschani, F.; Siebers, B.; Kaiser, M., Activity-based protein profiling as a robust method for enzyme identification and screening in extremophilic Archaea. Nat Commun 2017, 8, 15352.
26. Lentz, C. S.; Sheldon, J. R.; Crawford, L. A.; Cooper, R.; Garland, M.; Amieva, M. R.; Weerapana, E.; Skaar, E. P.; Bogyo, M., Identification of a S. aureus virulence factor by activity-based protein profiling (ABPP). Nat Chem Biol 2018, 14 (6), 609-617.
27. Niphakis, M. J.; Cravatt, B. F., Enzyme inhibitor discovery by activity-based protein profiling. Annu Rev Biochem 2014, 83, 341-77.
28. Liu, Y.; Patricelli, M. P.; Cravatt, B. F., Activity-based protein profiling: the serine hydrolases. Proc Natl Acad Sci USA 1999, 96 (26), 14694-9.
29. Li, S. W.; Yu, B.; Byrne, G.; Wright, M.; O'Rourke, S.; Mesa, K.; Berman, P. W., Identification and CRISPR/Cas9 Inactivation of the CIs Protease Responsible for Proteolysis of Recombinant Proteins Produced in CHO Cells. Biotechnol Bioeng 2019, 116 (9), 2130-2145.
30. Cravatt, B. F.; Wright, A. T.; Kozarich, J. W., Activity-based protein profiling: from enzyme chemistry to proteomic chemistry. Annu Rev Biochem 2008, 77, 383-414.
31. Wang, C.; Abegg, D.; Dwyer, B. G.; Adibekian, A., Discovery and Evaluation of New Activity-Based Probes for Serine Hydrolases. Chembiochem 2019, 20 (17), 2212-2216.
32. Adam, G. C.; Cravatt, B. F.; Sorensen, E. J., Profiling the specific reactivity of the proteome with non-directed activity-based probes. Chem Biol 2001, 8 (1), 81-95.
33. Adam, G. C.; Sorensen, E. J.; Cravatt, B. F., Proteomic profiling of mechanistically distinct enzyme classes using a common chemotype. Nat Biotechnol 2002, 20 (8), 805-9.
34. Yang, P.; Liu, K., Activity-based protein profiling: recent advances in probe development and applications. Chembiochem 2015, 16 (5), 712-24.
35. Bee, J. S.; Tie, L.; Johnson, D.; Dimitrova, M. N.; Jusino, K. C.; Afdahl, C. D., Trace levels of the CHO host cell protease cathepsin D caused particle formation in a monoclonal antibody product. Biotechnol Prog 2015, 31 (5), 1360-9.
36. Lim, A.; Doyle, B. L.; Kelly, G. M.; Reed-Bogan, A. M.; Breen, L. H.; Shamlou, P. A.; Lambooy, P. K., Characterization of a cathepsin D protease from CHO cell-free medium and mitigation of its impact on the stability of a recombinant therapeutic protein. Biotechnol Prog 2018, 34 (1), 120-129.
37. Luo, H.; Tie, L.; Cao, M.; Hunter, A. K.; Pabst, T. M.; Du, J.; Field, R.; Li, Y.; Wang, W. K., Cathepsin L Causes Proteolytic Cleavage of Chinese-Hamster-Ovary Cell Expressed Proteins During Processing and Storage: Identification, Characterization, and Mitigation. Biotechnol Prog 2019, 35 (1), e2732.
38. Repo, H.; Kuokkanen, E.; Oksanen, E.; Goldman, A.; Heikinheimo, P., Is the bovine lysosomal phospholipase B-like protein an amidase? Proteins 2014, 82 (2), 300-11.
39. Tjoelker, L. W.; Eberhardt, C.; Unger, J.; Trong, H. L.; Zimmerman, G. A.; McIntyre, T. M.; Stafforini, D. M.; Prescott, S. M.; Gray, P. W., Plasma platelet-activating factor acetylhydrolase is a secreted phospholipase A2 with a catalytic triad. J Biol Chem 1995, 270 (43), 25481-7.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

What is claimed is:

1. A method of identifying serine hydrolase in a biological test sample obtained from recombinant protein production, comprising the steps of:
a. adding a fluorophosphonate probe to the biological test sample comprising a serine hydrolase in conditions that allow the probe to bind to the serine hydrolase;
b. removing the free fluorophosphonate probes not bound to the serine hydrolase;
c. enriching the probe-bound serine hydrolases with an agent that binds to the probes;
d. digesting the serine hydrolase; and
e. identifying the serine hydrolase in the test sample with mass spectroscopy.

2. A method of identifying one or more serine hydrolases in a biological test sample causing PS-80 or PS-20 degradation, wherein the biological test sample is obtained from recombinant protein production, comprising the steps of:
a. adding a fluorophosphonate probe to the biological test sample that exhibits PS-80 or PS-20 degradation in conditions that allow the probe to bind to the serine hydrolase;
b. removing the free fluorophosphonate probes not bound to the serine hydrolase;
c. enriching the probe-bound serine hydrolases with an agent that binds to the probes;
d. digesting the serine hydrolase; and
e. identifying the one or more serine hydrolases in the test sample with mass spectroscopy.

3. The method of claim 1 or 2, wherein the agent in step c) is strepavidin or avidin beads.

4. The method of claim 1 or 2, wherein in step d) tyrpsin is used.

5. The method of claim 1, wherein the biological test sample is obtained from a CHO cell harvest cell culture fluid for antibody production, and optionally purified from one or more affinity, ion exchange, hydrophobic interaction, and mixed mode chromatography.

6. The method of claim 1 or 2, wherein the serine hydrolase is selected from the group consisting of SIAE (sialic acid acetylesterase), CES1 (liver carboxylesterase 1), PREP (prolyl endopeptidase), PLA2G7 (phospholipase A2, group VII), PRCP (prolylcarboxypeptidase), LPLA2, PLA1A (phospholipase A1 member A), LYPLA2 (lysophospholipase 2) and LYPLAl1 (lysophospholipase-like 1).

7. The method of any one of claims 1-2, wherein the serine hydrolase is selected from the group consisting of LPLA2, PLA2G7 (phospholipase A2, group VII), PLA1A (phospholipase A1 member A), LYPLA2 (lysophospholipase 2) and LYPLAl1 (lysophospholipase-like1).

8. The method of claim 1, wherein the flurophosphonate probe is 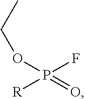
where R is alkyl, alkylheterocyclyl, —(CH₂)n₁-C(O)NH—(CH₂)n₂-NHC(O)(CH₂)n₃-R₁, —(CH₂)n₄-NHC(O)(CH₂)n₃-R₁, wherein n₁ is 8, 9, 10 or 11, n₂ is 2, 3, 4 or 5, n₃ is 2, 3, 4 or 5, and n₄ is 8, 9, 10 or 11; and R₁ is heterocyclyl.

9. The method of claim 1, wherein the flurophosphonate probe is 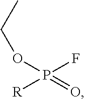
wherein R is —(CH₂)n₁-C(O)NH—(CH₂)n₂-NHC(O)(CH₂)n₃-R₁, wherein n₁ is 8, 9, 10 or 11, n₂ is 2, 3, 4, 5 or 6, n₃ is 2, 3, 4 or 5; R₁ is Heterocyclyl; and n₁+n₂+n₃=18.

10. The method of claim 1, wherein the flurophosphonate probe is 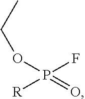
wherein R is —(CH₂)n₁-C(O)NH—(CH₂)n₂-NHC(O)(CH₂)n₃-R₁, wherein n₁ is 9, n₂ is 5, and n₃ is 4; and R₁ is

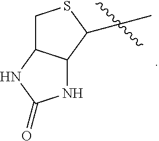 [SMILES: *C1SCC2NC(=O)NC21]

11. The method of claim 1, wherein the flurophosphonate probe is 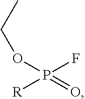
wherein R is —(CH₂)n₄-NHC(O)(CH₂)n₃-R₁, wherein n₃ is 5 and n₄ is 10; and
R₁ is

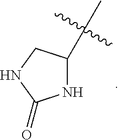 [SMILES: *C1CNC(=O)N1]

12. The method of claim 1, wherein the flurophosphonate probe is 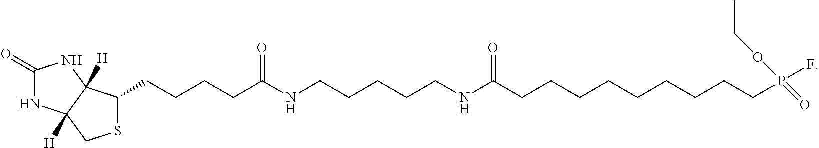 [SMILES: CCOP(=O)(F)CCCCCCCCCC(=O)NCCCCCNC(=O)CCCC[C@@H]1SC[C@@H]2NC(=O)N[C@@H]21]
